(19) United States
(12) Patent Application Publication
(10) Pub. No.: US 2022/0378823 A1
(43) Pub. Date: Dec. 1, 2022

(54) EXTRACORPOREAL TREATMENT FOR AGING

(71) Applicants: Arizona Board of Regents on behalf of Arizona State University, Scottsdale, AZ (US); Halberd Corporation, Jackson Center, PA (US)

(72) Inventors: Qiang Chen, Chandler, AZ (US); Mitchell Steven Felder, Hermitage, PA (US)

(21) Appl. No.: 17/825,132

(22) Filed: May 26, 2022

Related U.S. Application Data
(60) Provisional application No. 63/193,880, filed on May 27, 2021.

(51) Int. Cl. A61K 35/14; A61K 35/16; A61K 35/30; G01N 33/68; A61M 1/36

(52) U.S. Cl. CPC A61K 35/14; A61K 35/16; A61K 35/30; G01N 33/6845; A61M 1/3687

(57) ABSTRACT

An embodiment provides a method for treating a body fluid of a patient, including: removing the body fluid from a patient; applying a treatment to the body fluid, wherein the treatment comprises an antibody that joins with an aging process targeted antigen (TA) in the body fluid to form an antibody-TA complex, wherein the antibody comprises a tag sensitive to an illumination; removing the antibody-antigen complex from the body fluid; and returning the body fluid to the patient. Other aspects are described and claimed.

### CROSS REFERENCE TO RELATED APPLICATION

This application claims priority to U.S. Provisional Patent Application Ser. No. 63/193,880, filed on May 27, 2021, and entitled “EXTRACORPOREAL TREATMENT FOR AGING,” the contents of which are incorporated by reference herein.

### FIELD

This application relates generally to a treatment for aging in humans, and, more particularly, to an extracorporeal methodology for the treatment of aging.

### BACKGROUND

The aging process can be a major contributor to almost all known diseases. The aging process is especially intrinsic in the development of cancer, strokes, and neurodegenerative diseases. Effective treatments for biological processes that occur as humans get older may improve the length of life and improve the quality of life in older years. Slowing the aging process would be extremely useful in decreasing morbidity and mortality.

### BRIEF SUMMARY

In summary, one embodiment provides a method for treating a body fluid of a patient, comprising: removing the body fluid from a patient; applying a treatment to the body fluid, wherein the treatment comprises an antibody that joins with an aging process targeted antigen (TA) in the body fluid to form an antibody-TA complex, wherein the antibody comprises a tag sensitive to an illumination; removing the antibody-antigen complex from the body fluid; and returning the body fluid to the patient.

Another embodiment provides a device for treating a body fluid extracorporeally of a patient, comprising: a first stage including an inlet for the body fluid and at least one exterior wall defining a treatment chamber; a second stage, fluidly connected to the first stage, comprising a removal module and an outlet for the body fluid, wherein the treatment chamber comprises a delivery tube for introducing a treatment into the treatment chamber, wherein the delivery tube comprises a hollow tube including at least one interior wall defining a plurality of holes through which the treatment can be added to the treatment chamber, wherein the treatment is delivered through the hollow tube in counter-current mode with reference to the body fluid; the device being configured to: remove the body fluid from a patient; applying a treatment to the body fluid, wherein the treatment comprises an antibody that joins with an aging process targeted antigen (TA) in the body fluid to form an antibody-TA complex, wherein the antibody comprises a tag sensitive to an illumination; remove the antibody-antigen complex from the body fluid; and return the body fluid to the patient.

A further embodiment provides a product for treating a body fluid extracorporeally of a patient, comprising: a first stage including an inlet for the body fluid and at least one exterior wall defining a treatment chamber; a second stage, fluidly connected to the first stage, comprising a removal module and an outlet for the body fluid, wherein the treatment chamber comprises a delivery tube for introducing a treatment into the treatment chamber, wherein the delivery tube comprises a hollow tube including at least one interior wall defining a plurality of holes through which the treatment can be added to the treatment chamber, wherein the treatment is delivered through the hollow tube in counter-current mode with reference to the body fluid, wherein the treatment introduces antibodies selective to an antigen associated with an aging process.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example partial cross-sectional view of a cylinder and tubing used to deliver a treatment to a bodily fluid.

FIG. 2 illustrates an example a partial cross-sectional view showing additional detail of the cylinder and tubing of FIG. 1.

FIG. 3 illustrates an example flow diagram of a method for treatment of aging using extracorporeal methodology.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

In an embodiment, the method and system may extracorporeally treat a body fluid of a patient. The body fluid may be blood, cerebrospinal fluid (CSF), or the like. For ease of readability, blood may be used throughout the disclosure, however, other body fluids are contemplated and disclosed. The treatment includes a plurality of stages, comprising removing a bodily fluid or blood, hereinafter “blood,” from a patient, applying an extracorporeal treatment to the blood, and returning the blood to the patient.

In the first stage of the treatment, the body blood is removed from the patient. A convenient method for removing the blood is using standard venipuncture techniques. In the second stage, a treatment is applied to the blood. The treatment can include an antibody directed against a targeted antigen. The third stage comprises returning the blood to the patient and can also include removing the treatment from the blood before returning the blood to the patient.

In an embodiment, the method comprises treating a patient's blood extracorporeally with antibody(s) designed to react with particular targeted antigen (TA), including, but not limited to, mTOR (mammalian target of rapamycin), insulin growth factor-1 (IGF-1, insulin-like growth factor-1, somatomedin C), lipofuscin, p16 (p16INK4a, CDKN2A, cyclin-dependent kinase inhibitor 2A), SA-beta-gal (senescence-associated beta-gal), PML (promyelocytic leukemia) protein, TGF-β (transforming growth factor-beta), interleukin-6 (IL-6), indoleamine 2,3-dioxygenase, sTNF-R55 (soluble tumor necrosis factor-receptor 55), sTNF-R75 (soluble tumor necrosis factor-receptor 75), progerin, an oxygen-containing free radical (e.g., superoxide, nitric oxide, hydroxyl radical, peroxynitrite, nitrosoperoxycarbonate, hydrogen peroxide, hypochlorite), malondialdehyde (MDA, propanediol), tumor necrosis factor-α (TNF-alpha), and mitogen-activated protein kinases (MAPKs). The antibody may include a moiety, for example, an albumin moiety, that can complex with the target antigen/TA and thereby permit efficacious dialysis of the antibody-antigen complex. Dialysis methods are well-known by those of skill in the art.

In an embodiment, the antibody comprises a tag sensitive to an illumination. The treatment includes stages comprising removing the body fluid from a patient diagnosed with a process associated with aging, exposing and binding one or more combinations of those aging process Target Antigen(s) to fluorescent or luminous antibodies forming antibody complexes, eradicating the fluorescently or luminously antibody complexes with a laser, and then returning the body fluid to the patient

In an embodiment, the antibody comprises an albumin moiety and targets the removal of the TA from the blood, or body fluid.

The target antigen may be differentiated using standard enzyme-linked immunosorbent assay (ELISA) methodology. ELISA is a biochemical technique that allows the detection of an antigen in a sample. In ELISA, an antigen is affixed to a surface, and then an antibody is used for binding to the antigen. The antibody is linked to an enzyme that enables a color change in the substrate. Other strategies may be employed to validate the level of target antigen(s)/TA(s) in the body fluid, such as, but not limited to, Western blotting technology, UV/vis spectroscopy, mass spectrometry, and surface plasmon resonance (SPR).

In an embodiment, an alternative methodology may use a designer antibody with an attached macromolecular moiety instead of an albumin moiety. The macromolecular moiety, attached to the antibody, may be 1.000 mm to 0.00001 mm in diameter. The antibody-macromolecular moiety-targeted antigen complex may then be blocked from reentering the patient's blood, using a series of microscreens which contain openings with a diameter 50% to 99.99999% less than the diameter of the designer antibody-macromolecular moiety. The microscreen opening(s) may have a diameter of at least 25 μm to permit the passage and return to the circulation of the non-pathological blood constituents. Microscreen opening(s) and macromolecular diameters may be adjusted based upon a specific use.

Alternatively, the target antigen(s)/TA(s) may be captured using antibody microarrays, that contain antibodies to the target antigen(s). The antibody microarrays comprise millions of identical monoclonal antibodies attached at high density on glass or plastic slides. After sufficient extracorporeal exposure of the TA(s) to the antibody microarrays, the antibody microarrays-TA(s) may be disposed of, using standard medical practice.

In an embodiment, the method may comprise removing the targeted antigen(s)/TA(s) from the blood using a designer antibody containing an iron (Fe) moiety. This will then create a Fe-antibody-antigen complex. This iron-containing complex can then be efficaciously removed using a strong, localized magnetic force field.

In an embodiment, immunoaffinity chromatography may be used in which the heterogeneous group of molecules in the body fluid may undergo a purification process. There may be entrapment on a solid or stationary phase or medium. Only the targeted antigens (TAs) would be trapped using immunoaffinity chromatography. A solid medium may be removed from the mixture, washed, and the TA(s) may then be released from the entrapment through elution.

In an embodiment, gel filtration chromatography may be used in which the body fluid is used to transport the sample through a size-exclusion column that is used to separate the target antigen(s)/TA(s) by size and molecular weight.

Another alternative methodology would use molecular weight cut-off filtration. Molecular weight cut-off filtration may refer to the molecular weight at which at least 80% of the target antigen(s)/TA(s) is prohibited from membrane diffusion.

In an embodiment, the method and system may comprise at least three stages, including a first stage, a second stage, and a third stage. The first stage comprises removing blood from a patient. The second stage treats the blood. The thirds stage returns the treated blood to the patient after having achieved the physical removal of the targeted antigen, which can include mTOR (mammalian target of rapamycin), insulin growth factor-1 (IGF-1, insulin-like growth factor-1, somatomedin C), lipofuscin, p16 (p16INK4a, CDKN2A, cyclin-dependent kinase inhibitor 2A), SA-beta-gal (senescence-associated beta-gal), PML (promyelocytic leukemia) protein, TGF-beta (transforming growth factor-beta), interleukin-6 (IL-6), indoleamine 2,3-dioxygenase, sTNF-R55 (soluble tumor necrosis factor-receptor 55), sTNF-R75 (soluble tumor necrosis factor-receptor 75), progerin, an oxygen-containing free radical (e.g., superoxide, nitric oxide, hydroxyl radical, peroxynitrite, nitrosoperoxycarbonate, hydrogen peroxide, hypochlorite), malondialdehyde (MDA, propanediol), tumor necrosis factor-alpha (TNF-α), and mitogen-activated protein kinases (MAPKs), and combinations thereof.

The treatment may include the removal of the targeted antigen(s). The cleansed blood can then be returned to the patient, such as, for example, using the same catheter that was originally used in removing the blood. In one embodiment, the treatment of blood comprises removing 25 mL to 500 mL of blood from a patient, and then applying the treatment to the blood before returning it to the patient. The frequency of such treatments would depend upon an analysis of the underlying symptomatology and pathology of the patient.

In an embodiment, a short-duration pulse-beam from a laser or other high energy radiation emissive source may then be used to eradicate the aging process target antigen(s)-antibody complexes in the body fluid (blood plasma and/or blood), and then the body fluid may be returned back to the patient.

In an embodiment, in the first stage, a body fluid (e.g., blood plasma or blood) may be withdrawn from a patient using standard medical techniques. One convenient method for removing blood may be the standard venipuncture technique. Other techniques known to those skilled in the art are contemplated by this disclosure.

In an embodiment, in the second stage a treatment may be applied to a body fluid extracorporeally. The treatment may comprise exposing the body fluid to a fluorescent or luminous tagged antibody (F/LT Ab) generated to bind specific targeted antigens (TPAs) or targeted antigens (TAs) of aging processes such as those described above. During this treatment the fluorescently tagged or luminously conjugated antibody(s) and the targeted pathogen antigen form aging process fluorescent antibody complexes (F/LT Ab-TA complexes). Using an extremely narrow beam laser as an eradication tool, these aging process antibody complexes may be substantially eliminated from the extracorporeal body fluid. In an embodiment, the laser beam may be less than 10 nanometers in diameter.

In an embodiment, one method of eradication may include using an illumination system comprising an optic or other suitable sensor for detecting individual F/LT Ab-TA complexes in the extracorporeal body fluid. Additionally, in an embodiment, a high energy radiation source, such as a very narrow beam laser (for example, less than 10 nm in diameter), or another coherent light beam for eradicating the antibody complexes may be used. The body fluid may be pumped past the sensor where the body fluid may be illuminated by various techniques known in the art and the F/LT Ab-TAs can be identified by various techniques known in the art.

In an embodiment, a non-limiting generalized example is as follows: the sensor may be connected to a control unit. The signal from the sensed F/LT Ab-TA complexes may be transmitted to a control unit which controls a high energy emissive source. The receipt of a F/LT Ab-TA signal causes the control unit to emit a short-duration pulse-beam from a laser or other high energy radiation emissive source. The energy of the emitted radiation annihilates the F/LT Ab-TA, thereby destroying its disease-causing potential. The entire system may be monitored and controlled utilizing a computer, in real time, utilizing time units of 1 millisecond or less during the entire procedure. Persons having ordinary skill in art will recognize that the steps described above can be performed on various devices, machines, or systems. This disclosure contemplates all known devices, machines, or systems that can perform the steps described in the above illustrative example.

In an embodiment, the second stage substantially eliminates, through laser or other high-energy radiation emissive source targeting and annihilating, the F/LT Ab-TAs complexes from the body fluid (blood plasma and/or blood). In an embodiment, the laser, or other high-energy radiation emissive source, may not have a beam in excess of 10 nanometers in diameter. The illumination source may be computer directed and/or controlled in real time.

In an embodiment, the method includes removing blood from a patient in a first stage, treating the blood to obtain a reduction in the target antigen(s), and optionally removing the treatment from the blood in a second stage, and returning the blood to the patient in a third stage. The blood may be removed from the patient using any convenient method, including standard venipuncture procedure. The second stage may include sequentially passing the extracorporeal blood through a treatment chamber and a removal module.

In an embodiment, the second stage applies a treatment to the blood, which may include introducing a designer antibody that joins with a targeted antigen (TA) in the blood to form an antibody-antigen complex. The antibody-antigen complex may be removed from the blood in the removal module. Optionally, the antibody-antigen complex can be conjugated with a second antibody comprising a moiety that increases the efficacy of removal to form an antibody-moiety-antigen complex.

In a third stage, the purified blood, that is, blood that has been cleansed of the TA, can be returned to the patient.

In an embodiment, the device may include two stages. The first stage includes an inlet for the blood and at least one exterior wall defining a treatment chamber that is fluidly connected to a second stage. The second stage comprises a removal module and an outlet for the blood. In embodiments, the removal module can include, for example, a mechanical filter, a chemical filter, a dialysis machine, a magnet, a molecular filter, molecular adsorbent recirculating system (MARS), a plasmapheresis unit, and combinations thereof.

In an embodiment, a device for performing the method of the disclosure comprises a first stage including an inlet for blood and at least one exterior wall defining a treatment chamber that is fluidly connected to a second stage comprising a removal module and an outlet for the blood. The treatment chamber may include a delivery tube for introducing a treatment into the treatment chamber. In embodiments, the delivery tube comprises a hollow tube including at least one interior wall defining a plurality of holes through which the treatment may be added to the treatment chamber. The treatment may also be delivered through the hollow tube in counter-current mode with reference to the flow of the extracorporeal blood. The removal module may be any device capable of removing the antibody-antigen complex.

In an example, the first stage of the device applies a treatment of an antibody with an attached albumin moiety that targets the antigen. The second stage may facilitate substantial removal of the treatment from the extracorporeal blood.

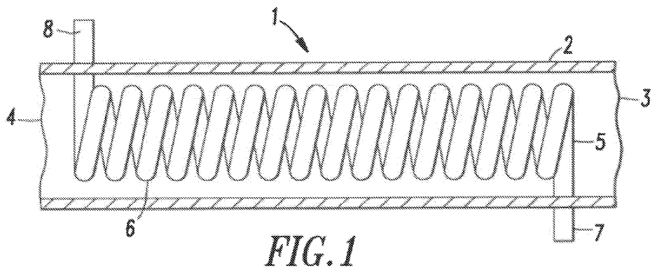

Referring to FIG. 1, in an embodiment, the first stage can include an exterior wall 2 defining a treatment chamber 5. The treatment can be applied in the treatment chamber 5. Residence times of the blood can be altered by changing the dimensions of the treatment chamber or the flow rate of the blood through the treatment chamber 5. Blood enters the inlet 3, passes through the treatment chamber 5, and exits the outlet 4. In embodiments, the treatment can be applied from a delivery tube 6 located within the treatment chamber 5. An interior wall 9 defines the delivery tube 6. The delivery tube 6 can include at least one lead 7, 8. The lead 7, 8 can deliver the treatment to the treatment chamber 5. Conveniently, the delivery tubes 6 may have a high contact surface area with the blood. As shown, the delivery tube 6 comprises a helical coil.

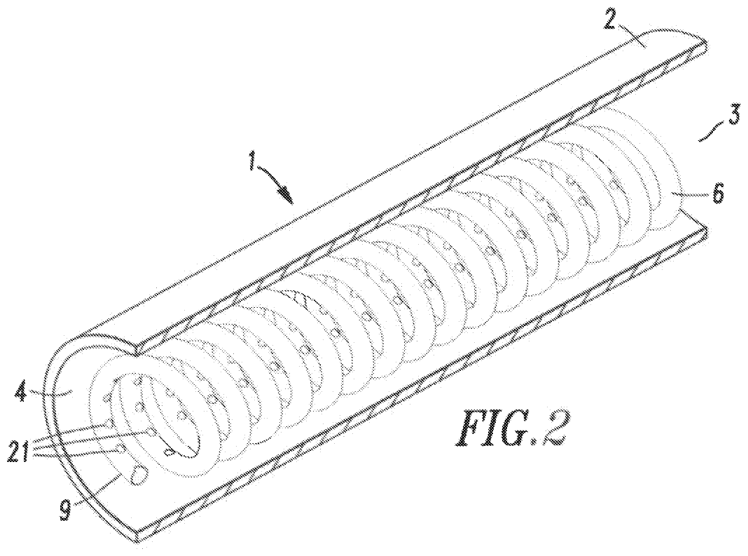

Referring to FIG. 2, in an embodiment, when the treatment includes the administration of a designer antibody, the delivery tube 6 can be hollow and the interior wall 9 may define a plurality of holes 21. The designer antibodies may be pumped through the delivery tube 6 to affect a desired concentration of designer antibodies in the blood. The designer antibodies may perfuse through the holes 21. The delivery tube 6 may include any suitable material including, for example, metal, plastic, ceramic or combinations thereof. The delivery tube 6 may also be rigid or flexible. In one embodiment, the delivery tube 6 may be a metal tube perforated with a plurality of holes. Alternatively, the delivery tube 6 may be plastic.

In an embodiment, the antibody with attached albumin moiety, targeting the antigen, may be delivered in a concurrent or counter-current mode with reference to the blood. In counter-current mode, the blood enters the treatment chamber 5 at the inlet 3. The designer antibody can enter through a first lead 8 near the outlet 4 of the treatment chamber 5. Blood then passes to the outlet 4 and the designer antibodies pass to the second lead 7 near the inlet 3. The removal module of the second stage substantially removes the designer antibodies-antigen molecular compound from the blood.

The second stage may include a filter, such as a dialysis machine, which is known to one skilled in the art. The second stage may include a molecular filter, for example, a molecular adsorbents recirculating system (MARS), which may be compatible and/or synergistic with dialysis equipment. MARS technology may be used to remove small-to-average-sized molecules from the blood. For example, artificial liver filtration presently uses this technique.

The methodology may include a plurality of steps for removing the targeted antigen. A first step may include directing a first antibody against the targeted antigen. A second step may include a second antibody. The second antibody can be conjugated with albumin, or alternatively a moiety that allows for efficacious dialysis. The second antibody or antibody-albumen complex combines with the first antibody forming an antibody-antibody-moiety complex. A third step may then be used to remove the complex from the blood. This removal may be enabled using dialysis and/or MARS. The purified blood may then be returned to the patient.

In an embodiment, a portion of the purified blood may be tested to ensure a sufficient portion of the targeted antigen has been successfully removed from the blood. Testing may determine the length of treatment and evaluate the efficacy of the sequential dialysis methodology in removing the targeted antigens. Blood with an unacceptably large concentration of complex remaining may then be re-filtered before returning the blood to the patient and/or adjustment to the method may be made to remove the complex.

In an embodiment, the second stage may remove the antibody-moiety-targeted antigen complex by techniques including, for example, filtering based on molecular size, protein binding, solubility, chemical reactivity, and combinations thereof. For example, a filter may include a molecular sieve, such as zeolite, or porous membranes that capture complexes comprising molecules above a certain size. Membranes may comprise polyacrylonitrile, polysulfone, polyamides, cellulose, cellulose acetate, polyacrylates, polymethylmethacrylates, and combinations thereof. Increasing the flow rate or dialysate flow rate may increase the rate of removal of the antibody with an attached albumin moiety targeting the antigen.

Another embodiment may include continuous renal replacement therapy (CRRT), which may remove large quantities of filterable molecules from the extracorporeal body fluid. CRRT would be particularly useful for molecular compounds that are not strongly bound to plasma proteins. Categories of CRRT include continuous arteriovenous hemofiltration, continuous venovenous hemofiltration, continuous arteriovenous hemodiafiltration, slow continuous filtration, continuous arteriovenous high-flux hemodialysis, and continuous venovenous high flux hemodialysis.

The sieving coefficient (SC) may be the ratio of the molecular concentration in the filtrate to the incoming blood. A SC close to zero indicates that the moiety antibody-targeted antigen complex will not pass through the filter. A filtration rate of about 10 mL per min is generally satisfactory. Other methods of increasing the removability of the moiety-antibody-targeted antigen include the use of temporary acidification of the blood using organic acids to compete with protein binding sites.

In an embodiment, a treatment is applied to a body fluid extracorporeally. The treatment comprises exposing the body fluid to a tagged antibody generated to bind specific targeted pathogenic antigens (TPAs) of the Covid-19 virus, or other target, such as those described above. During this treatment the conjugated antibody(s) and the targeted pathogen antigen form antibody complexes. A method for enhancing radiofrequency (RF) absorption includes providing targeted RF enhancers, such as antibodies with an attached RF absorption enhancer, such as, for example metal particles. The antibodies target and bind to the target to be removed from the body fluid. Binding RF enhancing particles to the antibodies (and other carriers having at least one targeting moiety) permits the injection of the antibodies (and other carriers having at least one targeting moiety) into the extracorporeal target solution. The RF enhancers induce the absorption of energy in the antibody-RF enhancing moiety complex. In addition, a combination of antibodies (and other carriers having at least one targeting moiety bound to different metals (and other RF absorbing particles) can be used allowing for variations in the RF absorption characteristics in the extracorporeal target area. The energy of the emitted radiofrequency (RF) annihilates the antibody-RF enhancing moiety complex, thereby destroying its disease-causing potential. The entire system is monitored and controlled utilizing a computer, in real time, utilizing time units of 1 millisecond or less during the entire procedure. Persons having ordinary skill in art will recognize that the steps described above can be performed on various devices/machines. This disclosure contemplates all known devices/machine that can perform the steps described in the above illustrative example.

A second stage substantially eliminates, through a high-energy radiofrequency emissive source targeting and annihilating, the antibody-RF enhancing moiety complex in the body fluid. A method for destroying a complex, or other virus or bacteria, is by introducing into the extracorporeal patient body fluid (blood, blood plasma, or CSF) RF absorption enhancers capable of selectively binding to the target virions and further capable of generating sufficient heat to kill or damage the bound target antibody-virion complexes by heat generated solely by the application of an RF field generated by an RF signal between a transmission head and a reception head.

Embodiments may include a method for slowing aging comprising: removing a body fluid from a patient; applying a treatment to the body fluid that targets an antigen associated with aging; and returning the body fluid to the patient in a third stage. Embodiments of the present invention also include such a method wherein the method includes removing the treatment from the body fluid.

Embodiments may include such a method wherein the antigen is selected from a group consisting of mTOR, insulin growth factor-1, lipofuscin, p16, SA-beta-gal, PML protein, TGF-beta, interleukin-6, indoleamine-2,3-dioxygenase, sTNF-R55, sTNF-R75, progerin, an oxygen-containing free radical, malondialdehyde, tumor necrosis factor-alpha, mitogen-activated protein kinase, and combinations thereof.

Embodiments may also include such a method wherein the treatment includes introducing an antibody that joins with the antigen to form an antibody-antigen complex; and removing the complex from the body fluid.

Embodiments may also include such a method wherein the treatment includes introducing a targeted antibody that combines with the antigen to form an antibody-antigen complex; and conjugating the antibody-antigen complex with a second antibody comprising a moiety that increases the efficacy of removal to form an antibody-moiety-antigen complex. Embodiments of the present invention also include such a method wherein the method includes testing the body fluid after the treatment and before returning the body fluid to the patient in order to determine efficacy.

An embodiment may include a method for slowing aging comprising: removing a body fluid from a patient; applying a treatment to the body fluid that targets an antigen associated with aging; and returning the body fluid to the patient in a third stage. For example, wherein the method includes removing the treatment from the body fluid. For example, wherein the antigen is selected from the group consisting of mTOR, insulin growth factor-1, lipofuscin, p16, SA-beta-gal, PML protein, TGF-beta, interleukin-6, indoleamine 2,3-dioxygenase, sTNF-R55, sTNF-R75, progerin, an oxygen-containing free radical, malondialdehyde, tumor necrosis factor-alpha, mitogen-activated protein kinases, and combinations thereof. For example, wherein the treatment includes: introducing an antibody that joins with the antigen to form an antibody-antigen complex; and removing the complex from the body fluid. For example, wherein the treatment includes: introducing a targeted antibody that combines with the antigen to form an antibody-antigen complex; and conjugating the antibody-antigen complex with a second antibody comprising a moiety that increases the efficacy of removal to form an antibody-moiety-antigen complex. For example, wherein the method includes testing the body fluid after the treatment and before returning the body fluid to the patient in order to determine efficacy.

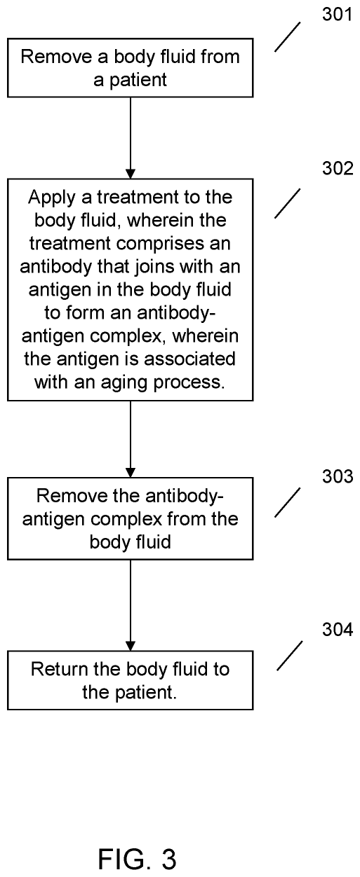

Referring to FIG. 3, an example method is illustrated. A method for treating a body fluid comprising: a first stage including removing the body fluid from a patient at 301; a second stage including applying a treatment to the body fluid by introducing an antibody that joins with an antigen in the body fluid to form an antibody-antigen complex at 302, and removing the antibody-antigen complex from the body fluid at 303; and a third stage including returning the body fluid to the patient at 304.

Unless otherwise indicated, all numbers expressing quantities of ingredients, properties such as molecular weight, reaction conditions, and so forth used in the specification and claims are to be understood as being modified in all instances by the term “about.”

Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

While the invention has been particularly shown and described with reference to a preferred embodiment, it will be understood by those skilled in the art that various changes in form and detail may be made therein without departing from the spirit and scope of the invention. All documents, books, manuals, papers, patents, published patent applications, guides, abstracts, and other references cited herein are incorporated by reference in their entirety.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data.

Embodiments may be implemented as an instrument, system, method or program product. Accordingly, an embodiment may take the form of an entirely hardware embodiment, or an embodiment including software (including firmware, resident software, micro-code, etc.) that may all generally be referred to herein as a “circuit,” “module” or “system.” Furthermore, embodiments may take the form of a program product embodied in at least one device readable medium having device readable program code embodied thereon.

A combination of device readable storage medium(s) may be utilized. In the context of this document, a device readable storage medium (“storage medium”) may be any tangible, non-signal medium that can contain or store a program comprised of program code configured for use by or in connection with an instruction execution system, apparatus, or device. For the purpose of this disclosure, a storage medium or device is to be construed as non-transitory, i.e., not inclusive of signals or propagating media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a hand held measurement device, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method for treating a body fluid of a patient, comprising:
removing the body fluid from a patient;
applying a treatment to the body fluid, wherein the treatment comprises an antibody that joins with an aging process targeted antigen (TA) in the body fluid to form an antibody-TA complex, wherein the antibody comprises a tag sensitive to an illumination;
removing the antibody-antigen complex from the body fluid; and
returning the body fluid to the patient.

2. The method of claim 1, wherein the aging process targeted antigen is selected from the group consisting of: mTOR, insulin growth factor-1, lipofuscin, p16, SA-beta-gal, PML protein, TGF-beta, interleukin-6, indoleamine-2,3-dioxygenase, sTNF-R55, sTNF-R75, progerin, an oxygen-containing free radical, malondialdehyde, tumor necrosis factor-alpha, and mitogen-activated protein kinase.

3. The method of claim 1, wherein the tag is selected from the group consisting of: a fluorescent tag and a luminous tag.

4. The method of claim 1, wherein illumination source comprises a narrow beam laser with a beam less than 10 nanometers in diameter.

5. The method of claim 1, wherein the illumination source annihilates the antibody-target antigen (TA) complex and a disease-causing potential of the target antigen.

6. The method of claim 1, wherein energy of an emitted radiation of the illumination source annihilates the antibody-target antigen (TA) complex.

7. The method of claim 1, further comprising capturing the antibody-target antigen (TA) complex using an antibody microarray.

8. The method of claim 1, further comprising determining an efficacy of treatment, using the illumination source, based on testing the body fluid after the treating the body fluid and before returning the body fluid to the patient.

9. The method of claim 1, wherein the removal further comprises a sensor for detecting individual antibody-target antigen complexes.

10. The method of claim 1, wherein the body fluid is selected from the group consisting of: blood plasma, blood, and cerebrospinal fluid.

11. A device for treating a body fluid extracorporeally of a patient, comprising:
a first stage including an inlet for the body fluid and at least one exterior wall defining a treatment chamber;
a second stage, fluidly connected to the first stage, comprising a removal module and an outlet for the body fluid, wherein the treatment chamber comprises a delivery tube for introducing a treatment into the treatment chamber, wherein the delivery tube comprises a hollow tube including at least one interior wall defining a plurality of holes through which the treatment can be added to the treatment chamber, wherein the treatment is delivered through the hollow tube in counter-current mode with reference to the body fluid;
the device being configured to:
remove the body fluid from a patient;
applying a treatment to the body fluid, wherein the treatment comprises an antibody that joins with an aging process targeted antigen (TA) in the body fluid to form an antibody-TA complex, wherein the antibody comprises a tag sensitive to an illumination;
remove the antibody-antigen complex from the body fluid; and
return the body fluid to the patient.

12. The device of claim 11, wherein the aging process targeted antigen is selected from the group consisting of: mTOR, insulin growth factor-1, lipofuscin, p16, SA-beta-gal, PML protein, TGF-beta, interleukin-6, indoleamine-2,3-dioxygenase, sTNF-R55, sTNF-R75, progerin, an oxygen-containing free radical, malondialdehyde, tumor necrosis factor-alpha, and mitogen-activated protein kinase.

13. The device of claim 11, wherein the tag is selected from the group consisting of: a fluorescent tag and a luminous tag.

14. The device of claim 11, wherein illumination source comprises a narrow beam laser with a beam less than 10 nanometers in diameter.

15. The device of claim 11, wherein the illumination source annihilates the antibody-target antigen (TA) complex and a disease-causing potential of the target antigen.

16. The device of claim 11, wherein energy of an emitted radiation of the illumination source annihilates the antibody-target antigen (TA) complex.

17. The device of claim 11, further comprising capturing the antibody-target antigen (TA) complex using an antibody microarray.

18. The device of claim 11, further comprising determining an efficacy of treatment, using the illumination source, based on testing the body fluid after the treating the body fluid and before returning the body fluid to the patient.

19. The device of claim 11, wherein the removal further comprises a sensor for detecting individual antibody-target antigen complexes.

20. A product for treating a body fluid extracorporeally of a patient, comprising:
a first stage including an inlet for the body fluid and at least one exterior wall defining a treatment chamber;
a second stage, fluidly connected to the first stage, comprising a removal module and an outlet for the body fluid, wherein the treatment chamber comprises a delivery tube for introducing a treatment into the treatment chamber, wherein the delivery tube comprises a hollow tube including at least one interior wall defining a plurality of holes through which the treatment can be added to the treatment chamber, wherein the treatment is delivered through the hollow tube in counter-current mode with reference to the body fluid, wherein the treatment introduces antibodies selective to an antigen associated with an aging process.
